(19) United States
(12) Patent Application Publication
(10) Pub. No.: US 2025/0375118 A1
(43) Pub. Date: Dec. 11, 2025

(54) WEARABLE DISPLAY DEVICE

(71) Applicant: Samsung Display Co., LTD., Yongin-si (KR)

(72) Inventor: Won Sang PARK, Yongin-si (KR)

(21) Appl. No.: 19/004,408

(22) Filed: Dec. 29, 2024

(30) Foreign Application Priority Data
Jun. 7, 2024 (KR) 10-2024-0074166

(51) Int. Cl. A61B 5/024; A61B 5/00; G06F 3/01; G06F 3/042; G08C 17/02; G09G 3/3233; H01Q 1/22

(52) U.S. Cl. CPC A61B 5/02438; A61B 5/0004; A61B 5/02427; A61B 5/6802; A61B 5/742; A61B 5/748; G06F 3/014; G06F 3/0421; H01Q 1/22; G08C 17/02; G09G 3/3233; G09G 2300/0819; G09G 2300/0842; G09G 2354/00; G09G 2360/14; G09G 2380/08

(57) ABSTRACT

A wearable display device includes: a housing formed in a ring shape or a cylindrical shape, a display panel disposed in a ring shape or a cylindrical shape inside the housing, and for emitting light in a first direction from an inner side of the display panel and receiving light incident to the inner side to detect a first light-sensing signal and for emitting light in a second direction from an outer side of the display panel and receiving light incident to the outer side to detect a second light-sensing signal, a main driver circuit for detecting a pulse wave signal of a user and measuring biometric information of the user by using the first light-sensing signal, and sensing the movement of a touch by a part of the user's body by using the second light-sensing signal, and a wireless communication module for transmitting the biometric information of the user.

This application claims priority to Korean Patent Application No. 10-2024-0074166, filed on Jun. 7, 2024, and all the benefits accruing therefrom under 35 U.S.C. § 119, the content of which in its entirety is herein incorporated by reference.

### BACKGROUND

### 1. Field of the Disclosure

The present disclosure relates to a wearable display device that can measure and transmit biometric information.

### 2. Description of the Related Art

As the information-oriented society evolves, various demands for display devices are ever increasing. Display devices are being employed by a variety of electronic devices such as smart phones, digital cameras, laptop computers, table PCs, navigation devices, and smart televisions. Portable display devices such as smartphones and tablet PCs may be equipped with a variety of features including image capturing, fingerprint recognition, face recognition, etc.

Recently, as the healthcare industry emerges as an industry with great prospects, methods for acquiring biometric information on health more conveniently are being developed. For example, there is an attempt to apply a traditional oscillometric blood pressure measurement device to a portable blood pressure measurement device. Such a portable blood pressure measurement device itself requires a separate light source, a sensor and a display, and is carried by a user in addition to a portable smart phone or tablet PC, which is inconvenient.

Recently, efforts have been made to combine portable display devices such as smartphones and tablet PCs with portable blood pressure measurement devices. Besides, there is a need for a method of measuring various biometric information such as heart rate, heart rate variability, respiration, cardiovascular disease and oxygen saturation in addition to blood pressure using portable display devices such as wearable display devices.

### SUMMARY

Aspects of the present disclosure provide a wearable display device that can be worn on a user's body in a ring or cylindrical shape, can measure biometric information such as blood pressure, and heart rate, and can wirelessly transmit the measured biometric information to other devices.

Aspects of the present disclosure also provide a wearable display device that can detect the user's biometric information using internal reflected light and sense the direction of the user's touch movement using external reflected light, by emitting light on both sides of the inner side and the outer side and sensing reflected light of the user's body from the both sides.

It should be noted that aspects of the present disclosure are not limited to the above-mentioned aspect; and other aspects of the present disclosure will be apparent to those skilled in the art from the following descriptions.

According to an embodiment of the disclosure, a wearable display device includes a housing formed in a ring shape or a cylindrical shape, a display panel disposed in a ring shape or a cylindrical shape inside the housing, and configured to emit light in a first direction from an inner side of the display panel and receive light incident to the inner side to detect a first light-sensing signal and configured to emit light in a second direction from an outer side of the display panel and receive light incident to the outer side to detect a second light-sensing signal, a main driver circuit configured to detect a pulse wave signal of a user and measure biometric information of the user by using the first light-sensing signal, and sense a movement of a touch by a part of the user's body by using the second light-sensing signal, and a wireless communication module configured to transmit the biometric information of the user to a preset other mobile display device through antennas formed in a preset pattern in the display panel.

According to an embodiment of the disclosure, a wearable electric display device includes a housing formed in a ring shape or a cylindrical shape, a display panel disposed in a ring shape or a cylindrical shape inside the housing, and configured to emit light in a first direction from an inner side of the display panel and receive light incident to the inner side to detect a first light-sensing signal and configured to emit light in a second direction from an outer side of the display panel and receive light incident to the outer side to detect a second light-sensing signal, a main driver circuit configured to detect a pulse wave signal of a user and measure biometric information of the user by using the first light-sensing signal, and sense a movement of a touch by a part of the user's body by using the second light-sensing signal, a wireless communication module configured to transmit the biometric information of the user to a preset other mobile display device through antennas formed in a preset pattern in the display panel, a circuit board on which the main driver circuit is mounted, the circuit board electrically connected to the display panel, a sensing circuit disposed in the housing or the circuit board to sense a direction of the user's movement and a pressure applied by the user's finger, and a memory storing and transmitting the pulse wave signals of the user and the biometric information of the user.

According to the embodiments of the present disclosure, a wearable display device can be worn on a user's body in a ring or cylindrical shape like a ring, a bracelet, a watch, or a band, so that it is possible to quickly and accurately measure the user's biometric information such as blood pressure, and heart rate in real time. In addition, the biometric information measured in real time can be wirelessly transmitted to other devices.

In addition, according to the embodiments of the present disclosure, a wearable display device can sense the direction in which the user's touch moves by emitting lights on both the inner and outer surfaces to sense reflected lights on the both surfaces, as well as detecting the user's biometric information. Accordingly, the wearable display device can find more applications, such as a direction control device for indicating and changing directions.

It should be noted that effects of the present disclosure are not limited to those described above and other effects of the present disclosure will be apparent to those skilled in the art from the following descriptions.

### BRIEF DESCRIPTION OF THE DRAWINGS

The above and other aspects and features of the present disclosure will become more apparent by describing in detail embodiments thereof with reference to the attached drawings, in which:

FIG. 1 is a perspective view showing an application example of a wearable display device according to an embodiment of the present disclosure;

FIG. 2 is a cross-sectional view showing a cross-sectional structure of the wearable display device shown in FIG. 1;

FIG. 3 is a top view showing the display panel shown in FIG. 2 that is spread out according to a first embodiment;

FIG. 4 is a circuit diagram showing a first light-emitting pixel disposed in the biological signal detection area of FIG. 3;

FIG. 5 is a circuit diagram showing a first light-sensing pixel disposed in the biological signal detection area of FIG. 3;

FIG. 6 is a cross-sectional view schematically showing the cross-sectional structure taken along line I-I′ of FIG. 3;

FIG. 7 is a cross-sectional view specifically showing the cross-sectional structure taken along line I-I′ of FIG. 6;

FIG. 8 is a block diagram showing an antenna arrangement structure of area AA shown in FIG. 3;

FIG. 9 is a cross-sectional view of a first embodiment showing a cross-sectional structure of a first light-sensing pixel and an antenna shown in FIG. 3;

FIG. 10 is a cross-sectional view of a second embodiment showing a cross-sectional structure of a first light-sensing pixel and an antenna shown in FIG. 3;

FIG. 11 is a cross-sectional view of a third embodiment showing a cross-sectional structure of a first light-sensing pixel and an antenna shown in FIG. 3;

FIG. 12 is a top view showing the display panel shown in FIG. 2 that is spread out according to a second embodiment;

FIG. 13 is a top view showing the display panel shown in FIG. 2 that is spread out according to a third embodiment;

FIG. 14 is a top view showing the display panel shown in FIG. 2 that is spread out according to a fourth embodiment;

FIG. 15 is a graph for illustrating a method for calculating information about blood pressure among biometric information according to the embodiment;

FIG. 16 is a graph for illustrating a method of calculating information about heart rate and respiration among biometric information according to an embodiment;

FIG. 17 is a cross-sectional view schematically showing the cross-sectional structure of a second light-emitting pixel and a second light-sensing pixel disposed in the touch detection area of FIG. 3;

FIG. 18 is a cross-sectional view specifically showing the cross-sectional structure of the second light-emitting pixel and the second light-sensing pixel shown in FIG. 17; and

FIG. 19 is a view for illustrating a method for identifying the direction in which a user's touch moves on a wearable display device according to an embodiment.

### DETAILED DESCRIPTION

The present disclosure will now be described more fully hereinafter with reference to the accompanying drawings, in which preferred embodiments of the disclosure are shown. This disclosure may, however, be embodied in different forms and should not be construed as limited to the embodiments set forth herein. Rather, these embodiments are provided so that this disclosure will be thorough and complete, and will filly convey the scope of the disclosure to those skilled in the art.

It will also be understood that when a layer is referred to as being “on” another layer or substrate, it can be directly on the other layer or substrate, or intervening layers may also be present. The same reference numbers indicate the same components throughout the specification.

It will be understood that, although the terms “first,” “second,” etc. may be used herein to describe various elements, these elements should not be limited by these terms. These terms are only used to distinguish one element from another element. For instance, a first element discussed below could be termed a second element without departing from the teachings of the present disclosure. Similarly, the second element could also be termed the first element.

Each of the features of the various embodiments of the present disclosure may be combined or combined with each other, in part or in whole, and technically various interlocking and driving are possible. Each embodiment may be implemented independently of each other or may be implemented together in an association.

Hereinafter, embodiments of the present disclosure will be described with reference to the accompanying drawings.

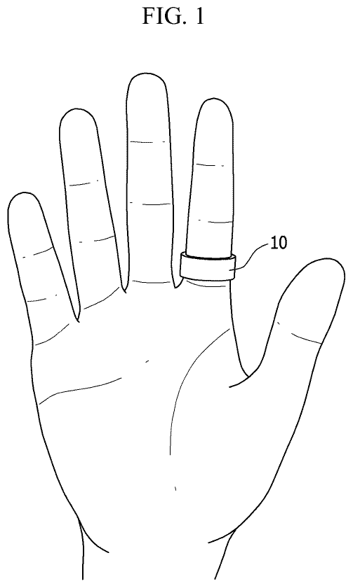

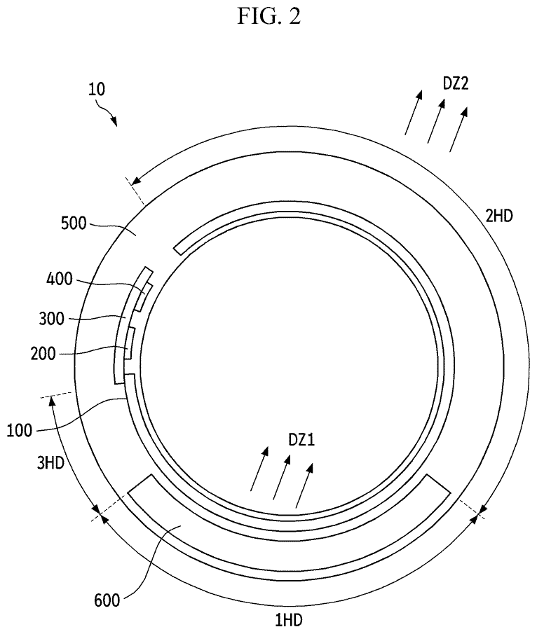

FIG. 1 is a perspective view showing an application example of a wearable display device according to an embodiment of the present disclosure. FIG. 2 is a cross-sectional view showing a cross-sectional structure of the wearable display device shown in FIG. 1.

Referring to FIGS. 1 and 2, a wearable display device 10 according to the embodiment is a ring or cylindrical type which can be worn on a user's body, like a ring, a bracelet, a smart watch, a band, a watch phone, etc.

The wearable display devices 10 may be divided into different light-emitting display devices such as an organic light-emitting display device using organic light-emitting diodes, a quantum-dot light-emitting display device including quantum-dot light-emitting layer, an inorganic light-emitting display device including an inorganic semiconductor, and a micro-LED display device using micro or nano light-emitting diodes (micro LEDs or nano LEDs) depending on the emission structure. In the following description, an organic light-emitting structure using an organic light-emitting diode is employed as the light-emitting structure of the wearable display device 10. It should be understood, however, that the embodiments of the present disclosure are not limited thereto.

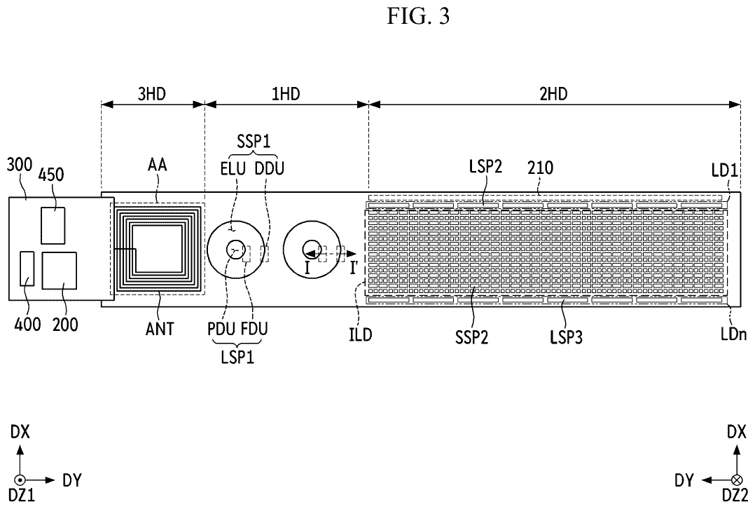

FIG. 3 is a top view showing the display panel shown in FIG. 2 that is spread out according to a first embodiment.

Referring to FIGS. 2 and 3, the wearable display device 10 according to the embodiment includes a display panel 100, a main driver circuit 200, a scan driver 210, a circuit board 300, a sensing circuit 450, a wireless communication module 400, and a housing 500.

The housing 500 is formed in a ring or cylindrical shape that may be applied to a ring, a bracelet, a watch, a band, etc. A plurality of grooves in which the display panel 100, the main driver circuit 200, the circuit board 300, etc., are embedded may be formed on the inner surface and the inside of the housing 500. The housing 500 may be made of opaque plastic, rubber, silicone, or metal material. The areas of the housing 500 corresponding to the light-emitting areas or light-receiving areas of the display panel 100 may be made of transparent glass or plastic.

The display panel 100 may be disposed in a ring shape or a cylindrical shape inside the housing 500. The display panel 100 may emit light and receive light in a first direction DZ1, i.e., toward the inside of the cylindrical housing 500 to detect a first light-sensing signal according to the amount of light reflected from the user's body. In addition, the display panel 100 may emit light and receive light in a second direction DZ2, i.e., toward the outside of the cylindrical housing 500 to detect a second light-sensing signal according to the amount of light reflected from the user's body.

The main driver circuit 200 may detect a user's pulse wave signal and measure the user's biometric information using the first light-sensing signal of the display panel 100, and may sense the movement of a touch by a part of the user's body using the second light-sensing signal. Specifically, the main driver circuit 200 may receive first light-sensing signals generated in the display panel 100 through light-sensing lines formed in the display panel 100, and may detect photoplethysmography signals, i.e., pulse wave signals, among the biological signals proportional to changes in the magnitude of the first light-sensing signals.

The main driver circuit 200 may analyze pulse wave signals every predetermined period and measure biometric information such as blood pressure, heart rate, heart rate variability, respiratory rate, blood vessel elasticity, cardiovascular disease, and oxygen saturation. The main driver circuit 200 may store the biometric information in the wireless communication module 400 and may transmit the biometric information stored in the wireless communication module 400 to another mobile display device, etc. through a separate cable, a communication circuit, an antenna, etc. The biometric information measurements such as blood pressure, heart rate, heart rate variability, respiratory rate, vascular elasticity, cardiovascular disease, and oxygen saturation may be displayed as an application program on the screen of the mobile display device.

The main driver circuit 200 may generate electric signals for driving the display panel 100 such as control signals and data voltages. The main driving circuit 200 may be implemented as an integrated circuit (IC) and may be attached to the display panel 10 or the circuit board 300 by a chip on glass (COG) technique, a chip on plastic (COP) technique, or an ultrasonic bonding. It is, however, to be understood that the present disclosure is not limited thereto. For example, the main driver circuit 200 may be attached on the circuit board 300 by the chip-on-film (COF) technique.

The scan driver 210 sequentially provides gate scan signals to the first and second light-emitting pixels and the first and second light-sensing pixels disposed in the display panel 100, thereby controlling the pixels so that the first and second light-emitting pixels emit lights and the first and second light-sensing pixels receive lights.

The scan driver 210 receives an emission control signal from the main driver circuit 200, and sequentially generates emission scan signals every horizontal line driving period in response to the emission control signal to sequentially provide them to the first and second light-emitting pixels. In other words, the scan driver 210 sequentially controls the emission timing of the first and second light-emitting pixels. In addition, the scan driver 210 may sequentially generate light-sensing scan signals in response to a scan control signal from the main driver circuit 200 and sequentially provide them to the first and second light-sensing pixels. That is to say, the scan driver 210 sequentially controls the light-sensing timing of the first and second light-sensing pixels.

The main driver circuit 200 is mounted on the circuit board 300, and the main driver circuit 200 is electrically connected to the display panel 100 through the circuit board 300. The circuit board 300 may be attached to one end of the display panel 100. Accordingly, the circuit board 300 may be electrically connected to the display panel 100 and the main driver circuit 200. The circuit board 300 may be a flexible printed circuit board, a printed circuit board, or a flexible film such as a chip-on film.

The sensing circuit 450 is disposed in the housing 500 or the circuit board 300 and senses the direction of the user's movement and the pressure applied by the user's finger. To this end, the sensing circuit 450 may include a motion detection sensor such as a gyro sensor and a pressure sensor. The sensing circuit 450 generates a motion detection signal in real time to transmit it to the main driver circuit 200, allowing the main driver circuit 200 to check the user's movement information in real time. In addition, the sensing circuit 450 transmits a pressure detection signal corresponding to a change in pressure applied by the user's finger to the main driver circuit 200.

The wireless communication module 400 stores the user's pulse wave signals and the user's biometric information generated in the main driver circuit 200 in the internal memory and the like. In addition, the user's pulse wave signals and the user's biometric information are transmitted to other mobile display devices, etc., through an antenna ANT patterned in the display panel 100.

Referring to FIG. 3, the display panel 100 may be divided into a biological signal detection area 1HD, a touch detection area 2HD, and an antenna placement area 3HD, which are not overlapped from each other.

At least one first light-emitting pixel SSP1 and at least one first light-sensing pixel LSP1 are formed and disposed in the biological signal detection area 1HD.

At least one first light-emitting pixel SSP1 emits light in the first direction DZ1, i.e., toward the inside of the display panel 100 disposed in a cylindrical shape, e.g., toward the inside of the cylindrical housing 500. The first light-emitting pixel SSP1 may be defined as a light-emitting pixel that is the minimum unit for emitting light of one of red, green, blue and white. For example, at least one first light-emitting pixel SSP1 may be formed as a light-emitting pixel that emits green light so that it can be used as a reflected light source for the vein of a finger or a wrist, among other body parts of the user.

At least one first light-emitting pixel SSP1 may include a light-emitting unit ELU that emits light in a first wavelength range, and a pixel driver unit DDU that applies a driving current to a light-emitting element of the light-emitting unit ELU. Herein, the light in the first wavelength range may be light in the green wavelength range. For example, the main peak wavelength of the light in the first wavelength range may be located approximately from 480 nm to 560 nm.

The pixel driver unit DDU applies a driving current to the light-emitting element of the light-emitting unit ELU in response to the data voltage from the main driver circuit 200 and the gate scan signal from the scan driver 210. The light-emitting element of the light-emitting unit ELU emits light in the green wavelength range by the amount of the driving current applied from the pixel driver unit DDU. The light-emitting element of the light-emitting unit ELU may be formed in a circular shape or a polygonal shape, such as a rectangle when viewed from the top, and may surround a light-sensing unit of the first light-sensing pixel LSP1.

At least one first light-sensing pixel LSP1 disposed in the biological signal detection area 1HD receives light reflected from a part of the user's body in the first direction DZ1 and generates a first light-sensing signal based on the amount of received light. The first light-sensing pixel LSP1 may be defined as a light-sensing pixel that is the minimum unit for outputting a light-sensing signal which varies depending on the amount of received light as a light-sensing signal.

At least one first light-sensing pixel LSP1 includes a light-sensing unit PDU that outputs a light-sensing current proportional to the amount of incident light in the first direction DZ1, and a sensing driver unit FDU that provides a first light-sensing signal proportional to the amount of the light-sensing current from the light-sensing unit PDU to the main driver circuit 200.

The light-sensing unit PDU may include a light-sensing element PD that generates and outputs a light-sensing current proportional to the amount of received light. The light-sensing element PD of the light-sensing unit PDU may be disposed adjacent to the light-emitting unit ELU of the first light-emitting pixel SSP1. The light-sensing element PD may be formed in a circular shape or a polygonal shape, such as a rectangle when viewed from the top and may be formed in a shape surrounded by the light-emitting unit ELU. The light-sensing element PD may be an organic photodiode (“OPD”) sensor. The light-sensing unit PDU may be formed with a transmittance from 80% to 100% and may detect light in a low brightness or low-illuminance range. To this end, only a transparent protective film or protective cover, etc., may be disposed on the front side of the light-sensing unit PDU. For example, the low brightness range may be set in advance to the range of 0.0005 cd/m² to 0.0001 cd/m², and the light-sensing unit PDU may be formed with a transmittance from 80% to 100% to detect light in the low brightness range.

The touch detection area 2HD of the display panel 100 includes a light exit area ILD, a first light-sensing area LD1, and the n^{th} light-sensing area LDn.

The light exit area ILD includes at least one second light-emitting pixel SSP2 that emits light in the second direction DZ2, i.e., toward the outside of the cylindrical housing 500. In the light exit area ILD, a plurality of second light-emitting pixels SSP2 may be arranged in vertical or horizontal stripes, or may be arranged the PenTile matrix.

Each of the second light-emitting pixels SSP2 arranged in a matrix in the light exit area ILD may emit light toward the outside of the display panel 100 in a cylindrical shape, e.g., in the second direction DZ2 toward the outside of the cylindrical housing 500 in FIG. 2. Each of the second light-emitting pixels SSP2 may be defined as a light-emitting pixel that is the minimum unit for emitting light of one of red, green, blue and white. For example, at least one second light-emitting pixel SSP2 may be formed as a light-emitting pixel that emits red or white light so that it can be used as a reflected light source for the skin of a finger or a palm, among other body parts of the user.

The first light-sensing area LD1 may be disposed on one side of the light exit area ILD. The first light-sensing area LD1 includes a plurality of second light-sensing pixels LSP2 that detects the amount of light reflected from a part of the user's body in the second direction DZ2 and generate a second light-sensing signal based on the detected amount of light. The second light-sensing pixels LSP2 formed in the first light-sensing area LD1 are arranged side-by-side on one side of the light exit area ILD along the longitudinal direction of the display panel 100 and the light exit area ILD.

The n^{th} light-sensing area LDn may be disposed on the opposite side of the light exit area ILD. The first light-sensing area LD1 includes a plurality of third light-sensing pixels LSP3 that detects the amount of light reflected from a part of the user's body in the second direction DZ2 and generate a third light-sensing signal based on the detected amount of light. The plurality of third light-sensing pixels LSP3 formed in the n^{th} light-sensing area LDn are arranged side-by-side on the opposite side of the light exit area ILD along the longitudinal direction of the display panel 100 and the light exit area ILD. Accordingly, the plurality of third light-sensing pixels LSP3 formed in the n^{th} light-sensing area LDn are arranged in parallel with the plurality of second light-sensing pixels LSP2 formed in the first light-sensing area LD1 with the light exit area ILD therebetween.

In the antenna placement area 3HD, at least one antenna ANT patterned in a preset pattern and a plurality of signal pads respectively connected to one end and the other end of the antenna ANT are formed. The antenna ANT is patterned in the antenna placement area 3HD of the display panel 100 in a zigzag shape, a coil shape, a block shape, or a combination of various preset shapes on a plane, and transmits electromagnetic waves including electric signals in respect to the pulse wave signals and biometric information. To this end, the wireless communication module 400 sequentially transmits the electric signals in respect to the pulse wave signals and biometric information of the user to the signal pads of the antenna ANT, and the antenna ANT converts the electric signals in respect to the pulse wave signals and biometric information of the user to electromagnetic waves and transmit them.

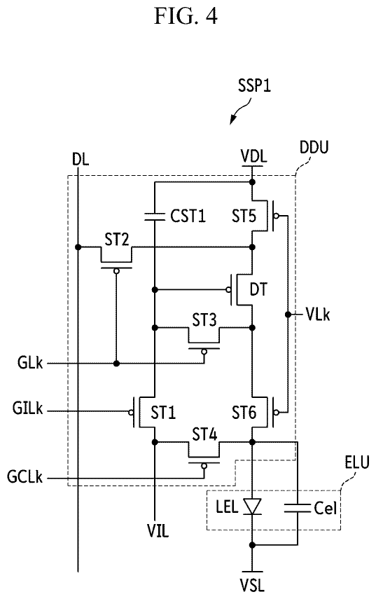

FIG. 4 is a circuit diagram showing a first light-emitting pixel disposed in the biological signal detection area of FIG. 3.

Referring to FIG. 4, each of the first light-emitting pixels SSP1 according to the embodiment may be connected to the k^{th} display initialization line GILk, the k^{th} display scan line GLk, and the k^{th} display control line GCLk, and the k^{th} emission control line VLk. In addition, the first light-emitting pixel SSP1 may be connected to a first driving voltage line VDL from which a first driving voltage is supplied, a second driving voltage line VSL from which a second driving voltage is supplied, and a third driving voltage line VIL from which a third driving voltage is supplied. In the following description, the letters such as k and n used in place of numbers are defined as positive integers excluding zero.

As described above, the first light-emitting pixel SSP1 may include the light-emitting unit ELU and the pixel driver unit DDU. The light-emitting unit ELU may include a light-emitting element LEL. The pixel driver unit DDU may include a driving transistor DT, switch elements, and a capacitor CST1. The switch elements include first to sixth transistors ST1, ST2, ST3, ST4, ST5 and ST6.

The driving transistor DT may include a gate electrode, a first electrode and a second electrode. A drain-source current Ids (hereinafter referred to as “driving current”) of driving transistor DT flowing between the first electrode and the second electrode is controlled according to the data voltage applied to the gate electrode. The driving current Ids flowing through the channel of the driving transistor DT is proportional to the square of the difference between a voltage Vgs between the first electrode and the gate electrode of the driving transistor DT and the threshold voltage, as shown in Equation 1 below:

$$Ids = {k}^{′} \times {( Vsg - Vth )}^{2} \qquad [ Equation 1 ]$$

where k′ denotes a proportional coefficient determined by the structure and physical properties of the driving transistor, Vsg denotes the voltage between the first electrode and the gate electrode of the driving transistor, and Vth denotes the threshold voltage of the driving transistor.

The light-emitting element LEL emits light as the driving current Ids flows therein. The amount of the light emitted from the light-emitting elements LEL may increase with the driving current Ids.

The light-emitting element LEL may be an organic light-emitting diode including an organic emissive layer disposed between an anode electrode and a cathode electrode. Alternatively, the light-emitting element LEL may be an inorganic light-emitting element including an inorganic semiconductor disposed between an anode electrode and a cathode electrode. Alternatively, the light-emitting element LEL may be quantum-dot light-emitting element including a quantum-dot emissive layer disposed between an anode electrode and a cathode electrode. Alternatively, the light-emitting element LEL may be a micro light-emitting element including a micro light-emitting diode disposed between an anode electrode and a cathode electrode.

The anode electrode of the light-emitting element LEL may be connected to the first electrode of the fourth transistor ST4 and the second electrode of the sixth transistor ST6, while the cathode electrode thereof may be connected to the second driving voltage line VSL. A parasitic capacitance Cel may be formed between the anode electrode and the cathode electrode of the light-emitting element LEL.

The first transistor ST1 is turned on by an initialization scan signal of the k^{th} display initialization line GILk to connect the gate electrode of the driving transistor DT with the third driving voltage line VIL. Accordingly, a third driving voltage VINT of the third supply voltage line VILI may be applied to the gate electrode of the driving transistor DT. The gate electrode of the first transistor ST1 may be connected to the k^{th} display initialization line GILk, the first electrode thereof may be connected to the gate electrode of the driving transistor DT, and the second electrode thereof may be connected to the third driving voltage line VIL.

The second transistor ST2 is turned on by the display scan signal of the k^{th} display scan line GLk to connect the first electrode of the driving transistor DT with the data line DL.

Accordingly, the data voltage of the data line DL may be applied to the first electrode of the driving transistor DT. The gate electrode of the second transistor ST2 may be connected to the k^{th} display scan line GLk, a first electrode thereof may be connected to the first electrode of the driving transistor DT, and a second electrode thereof may be connected to the data line DL.

The third transistor ST3 is turned on by the display scan signal of the k^{th} display scan line GLk to connect the gate electrode with the second electrode of the driving transistor DT. When the gate electrode and the second electrode of the driving transistor DT are connected with each other, the driving transistor DT works as a diode. A gate electrode of the third transistor ST3 may be connected to the k^{th} display scan line GLk, a first electrode thereof may be connected to the second electrode of the driving transistor DT, and a second electrode thereof may be connected to the gate electrode of the driving transistor DT.

The fourth transistor ST4 is turned on by a display control signal of the k^{th} display control line GCLk to connect the anode electrode of the light-emitting element LEL with the third driving voltage line VIL. The third driving voltage of the third driving voltage line VIL may be applied to the anode electrode of the light-emitting element LEL. The gate electrode of the fourth transistor ST4 may be connected to the k^{th} display control line GCLk, the first electrode thereof may be connected to the anode electrode of the light-emitting element LEL, and the second electrode thereof may be connected to the third driving voltage line VIL.

The fifth transistor ST5 is turned on by the emission signal of a k^{th} emission control line VLk to connect the first electrode of the driving transistor DT with the first driving voltage line VDL. The gate electrode of the fifth transistor ST5 is connected to the k^{th} emission control line VLk, the first electrode thereof is connected to the first driving voltage line VDL, and the second electrode thereof is connected to the first electrode of the driving transistor DT.

The sixth transistor ST6 is disposed between the second electrode of the driving transistor DT and the anode electrode of the light-emitting element LEL. The sixth transistor ST6 is turned on by the emission control signal of the k^{th} emission control line VLk to connect the second electrode of the driving transistor DT with the anode electrode of the light-emitting element LEL. The gate electrode of the sixth transistor ST6 is connected to the k^{th} emission control line VLk, the first electrode thereof is connected to the second electrode of the driving transistor DT, and the second electrode thereof is connected to the anode electrode of the light-emitting element LEL.

When both the fifth transistor ST5 and the sixth transistor ST6 are turned on, the driving current Ids of the driving transistor DT according to the data voltage applied the gate electrode of the driving transistor DT may flow to the light-emitting element LEL.

The capacitor CST1 is formed between the gate electrode of the driving transistor DT and the first driving voltage line VDL. The first capacitor electrode of the capacitor CST1 may be connected to the gate electrode of the driving transistor DT, and the second capacitor electrode thereof may be connected to the first driving voltage line VDL.

When the first electrode of each of the first to sixth transistors ST1, ST2, ST3, ST4, ST5 and ST6 and the driving transistor DT is a source electrode, the second electrode thereof may be a drain electrode. Alternatively, when the first electrode of each of the first to sixth transistors ST1, ST2, ST3, ST4, ST5 and ST6 and the driving transistor DT is a drain electrode, the second electrode thereof may be a source electrode.

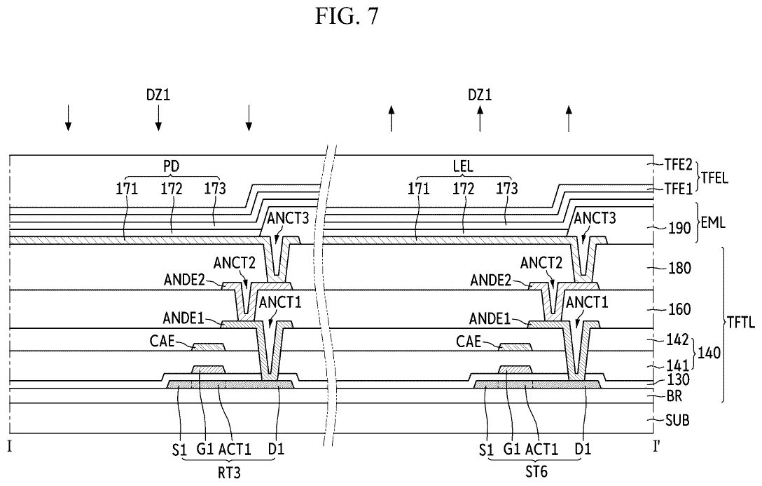

The active layer of each of the first to sixth transistors ST1, ST2, ST3, ST4, ST5 and ST6 and the driving transistor DT may be formed of one of poly silicon, amorphous silicon and oxide semiconductor. Although the first to sixth transistors ST1, ST2, ST3, ST4, ST5 and ST6 and the driving transistor DT are implemented as p-type metal oxide semiconductor field effect transistors (MOSFETs) in FIG. 7, this is merely illustrative. They may be implemented as n-type MOSFETs. For example, the first to sixth transistors ST1, ST2, ST3, ST4, ST5 and ST6, and the driving transistor DT may be implemented as n-type MOSFETs. Alternatively, at least one of the first to sixth transistors ST1, ST2, ST3, ST4, ST5 and ST6 may be implemented as an n-type MOSFET.

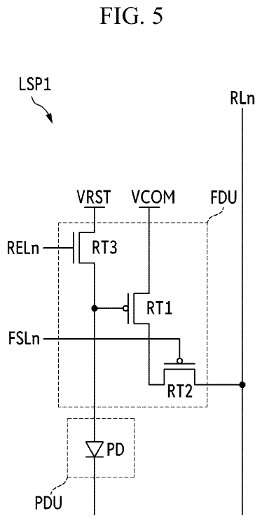

FIG. 5 is a circuit diagram showing a first light-sensing pixel disposed in the biological signal detection area of FIG. 3.

Referring to FIG. 5, the first light-sensing pixel LSP1 is electrically connected to the n^{th} sensing reset line RSLn, the n^{th} light-sensing scan line FSLn, and the n^{th} light-sensing line RLn. Each of the light-sensing pixels LSP may be reset by a reset signal from the n^{th} sensing reset line RSLn, and may transmit a light-sensing signal to the n^{th} light-sensing line RLn in response to the sensing scan signal from the n^{th} light-sensing scan line FSLn.

The first light-sensing pixels LSP1 may be divided into a light-sensing unit PDU including a light-sensing element PD, and a sensing driver unit FDU including first to third sensing transistors RT1 to RT3 and a sensing capacitor (not shown). The sensing capacitor may be formed in parallel with the light-sensing element PD.

A first sensing transistor RT1 of the sensing driver unit FDU may allow a light-sensing current to flow according to the voltages of the light-sensing element PD and the sensing capacitor. The amount of current of the light-sensing current may vary depending on a voltage applied to the light-sensing element PD and the sensing capacitor. The gate electrode of the first sensing transistor RT1 may be connected to the second electrode of the light-sensing element PD. A first electrode of the first sensing transistor RT1 may be connected to a common voltage source VCOM from which a common voltage is applied. A second electrode of the first sensing transistor RT1 may be connected to a first electrode of the second sensing transistor RT2.

When the sensing scan signal of the gate-on voltage is applied to the n^{th} light-sensing scan line FSLn, the second sensing transistor RT2 allows the sensing current of the first sensing transistor RT1 to flow to the n^{th} light-sensing line RLn. In this instance, the n^{th} light-sensing line RLn may be charged with the sensing voltage by the sensing current. The gate electrode of the second sensing transistor RT2 may be connected to the n^{th} light-sensing scan line FSLn, the first electrode thereof may be connected to the second electrode of the first sensing transistor RT1, and the second electrode thereof may be connected to the n^{th} light-sensing line RLn.

When a reset signal of the gate-on voltage is applied to the n^{th} sensing reset line RSLn, the third sensing transistor RT3 may reset the voltages of the light-sensing element PD and the sensing capacitor to the reset voltage of a reset voltage source VRST. The gate electrode of the third sensing transistor RT3 may be connected to the sensing reset line RSL, the first electrode thereof may be connected to the reset voltage source VRST, and the second electrode thereof may be connected to the second electrode of the light-sensing element PD.

Although the first sensing transistor RT1 and the second sensing transistor RT2 are implemented as p-type metal oxide semiconductor field effect transistors (MOSFETs) while the third sensing transistor RT3 is implemented as an n-type MOSFET in the example shown in FIG. 5, this is merely illustrative. Optionally, they may be of the same type or different types. In addition, one of the first and second electrodes of each of the first sensing transistor RT1, the second sensing transistor RT2 and the third sensing transistor RT3 may be a source electrode, while the other one may be a drain electrode.

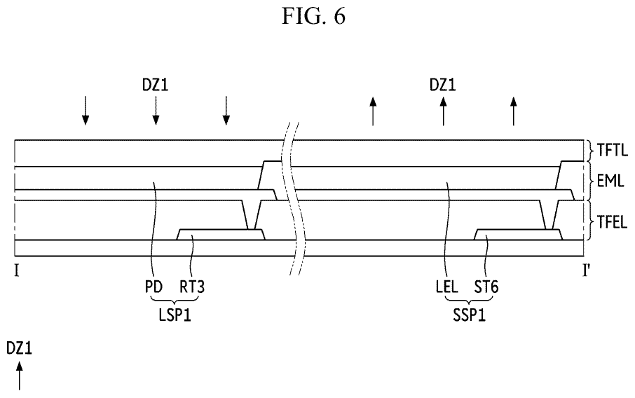

FIG. 6 is a cross-sectional view schematically showing the cross-sectional structure taken along line I-I′ of FIG. 3. FIG. 7 is a cross-sectional view specifically showing the cross-sectional structure taken along line I-I′ of FIG. 6.

First, referring to FIGS. 3 and 6, a light-sensing element PD of a light-sensing unit PDU formed in a first light-sensing pixel LSP1 may be formed in a circular shape or a polygonal shape such as a square when viewed from the top, and may be surrounded by a light-emitting unit ELU.

In addition, the light-emitting elements LEL formed in the light-emitting unit ELU of the first light-emitting pixel SSP1 may be formed in a circular shape or a polygonal shape such as a rectangle when viewed from the top, and may surround the light-sensing unit of the first light-sensing pixel LSP1.

Referring to FIGS. 6 and 7, a substrate SUB of the display panel 100 may be a base substrate or a base member. The substrate SUB may be a flexible substrate that can be bent, folded, or rolled. The substrate SUB may include a polymer resin such as polyimide PI.

A thin-film transistor layer TFEL may be disposed on the substrate SUB. The thin-film transistor layer TFEL may include a plurality of thin-film transistors forming a pixel driver unit DDU of the first light-emitting pixel SSP1 and a sensing driver unit FDU of a first light-sensing pixel LSP1. The thin-film transistor layer TFEL may further include gate lines, data lines, voltage lines, gate control lines, fan-out lines for connecting the main driver circuit 200 with the data lines, lead lines for connecting the main driver circuit 200 with the pads, etc. When the scan driver 210 is formed on one end of the display panel 100, the scan driver 210 may also include thin-film transistors.

An emission material layer EML may be disposed on the thin-film transistor layer TFEL. The emission material layer EML may include a plurality of light-emitting elements in each of which a first electrode, an organic material layer and a second electrode are stacked on one another sequentially to emit light, and a pixel-defining layer for defining an area in which each of light-emitting elements PD is to be formed.

An encapsulation layer TFTL may cover the upper and side surfaces of the emission material layer EML, and can protect the emission material layer EML. The encapsulation layer TFTL may include at least one inorganic layer and at least one organic layer for encapsulating the emission material layer EML.

Referring to FIG. 7, the barrier layer BR is a film for protecting the transistors of the thin-film transistor layer TFTL and an organic material layer 172 of the emission material layer EML. The barrier layer BR may be made up of multiple inorganic layers stacked on one another alternately.

The thin-film transistor ST6 and RT3, etc., may be disposed on the barrier layer BR. Each of the thin-film transistors ST6 and RT3 includes an active layer ACT1, a gate electrode G1, a source electrode S1 and a drain electrode D1.

The active layer ACT1, the source electrode SI and the drain electrode D1 of each of the thin-film transistors ST6 and RT3 may be disposed on the barrier layer BR. The active layer ACTI of each of the thin-film transistors ST6 and RT3 includes polycrystalline silicon, single crystalline silicon, low-temperature polycrystalline silicon, amorphous silicon, or an oxide semiconductor. A part of the active layer ACT1 overlapping the gate electrode G1 in the third direction (z-axis direction) that is the thickness direction of the substrate SUB may be defined as a channel region. The source electrode S1 and the drain electrode D1 are regions that do not overlap with the gate electrode G1 in the third direction (z-axis direction), and may have conductivity by doping ions or impurities into a silicon semiconductor or an oxide semiconductor.

A gate insulator 130 may be disposed on the active layer ACT1, the source electrode S1 and the drain electrode DI of each of the thin-film transistors ST6 and RT3.

The gate electrode G1 of each of the thin-film transistors ST6 and RT3 may be disposed on the gate insulator 130. The gate electrode G1 may overlap the active layer ACT1 in the third direction (z-axis direction). The gate electrode G1 may be made up of a single layer or multiple layers of one of molybdenum (Mo), aluminum (Al), chromium (Cr), gold (Au), titanium (Ti), nickel (Ni), neodymium (Nd) and copper (Cu) or an alloy thereof.

A first interlayer-dielectric layer 141 may be disposed on the gate electrode G1 of each of the thin-film transistors ST6 and RT3. The first interlayer-dielectric layer 141 may be made up of at least one inorganic layer, for example, silicon nitride layer, silicon oxynitride layer, silicon oxide layer, and the like.

A capacitor electrode CAE may be disposed on the first interlayer-dielectric layer 141. The capacitor electrode CAE may overlap with the gate electrode G1 of each of the thin-film transistors ST6 and RT3 in the third direction (z-axis direction). Since the first interlayer-dielectric layer 141 has a predetermined dielectric constant, a capacitor can be formed by the capacitor electrode CAE, the gate electrode G1, and the first interlayer-dielectric layer 141 disposed between them. A second interlayer-dielectric layer 142 may be disposed over the capacitor electrode CAE.

A first anode connection electrode ANDE1 may be disposed on the second interlayer-dielectric layer 142. The first anode connection electrode ANDE1 may be connected to the drain electrode D1 of each of the thin-film transistors ST6 and RT3 through a first connection contact hole ANCT1 that penetrates the gate insulator 130, the first interlayer-dielectric layer 141 and the second interlayer-dielectric layer 142. The first anode connection electrode ANDE1 may be made up of a single layer or multiple layers of one of molybdenum (Mo), aluminum (Al), chromium (Cr), gold (Au), titanium (Ti), nickel (Ni), neodymium (Nd) and copper (Cu) or an alloy thereof. A first planarization layer 160 may be disposed over the first anode connection electrode ANDE1 for providing a flat surface over the thin-film transistors ST6 and RT3.

A second anode connection electrode ANDE2 may be disposed on the first planarization layer 160. The second anode connection electrode ANDE2 may be connected to the first anode connection electrode ANDE1 through a second connection contact hole ANCT2 penetrating the first planarization layer 160. The second anode connection electrode ANDE2 may be made up of a single layer or multiple layers of one of molybdenum (Mo), aluminum (Al), chromium (Cr), gold (Au), titanium (Ti), nickel (Ni), neodymium (Nd) and copper (Cu) or an alloy thereof. A second planarization layer 180 may be disposed on the second anode connection electrode ANDE2.

Light-emitting elements LEL, light-sensing elements PD and a pixel-defining layer 190 may be disposed on the second planarization layer 180. Each of the light-emitting elements LEL and the light-sensing elements PD includes a pixel electrode 171, an organic material layer 172, and a common electrode 173. An organic material made of an organic light-emitting material may be formed in the organic material layer 172 of the light-emitting elements LEL. In the emission area of the light-emitting elements LEL, the pixel electrode 171, the organic material layer 172 and the common electrode 173 are stacked on one another sequentially, so that holes from the pixel electrode 171 and electrons from the common electrode 173 are combined with each other in the organic material layer 172 to emit light.

The organic material layer 172 of the light-emitting element LEL formed in the light-emitting unit ELU of the first light-emitting pixel SSP1 may be formed in a circular shape or a polygonal shape such as a rectangle when viewed from the top, and may surround the light-sensing element PD of the first light-sensing pixel LSP1. On the other hand, the organic material layer 172 of the light-sensing element PD formed in the first light-sensing pixel LSP1 may be formed in a circular shape or a polygonal shape such as a rectangle when viewed from the top, and may be surrounded by the light-emitting element LEL.

In the top-emission structure in which light exits from the organic material layer 172 toward the common electrode 173, the pixel electrode 171 may be made of a metal material having a high reflectivity such as a stack structure of aluminum and titanium (Ti/Al/Ti), a stack structure of aluminum and indium tin oxide (ITO) (ITO/Al/ITO), an APC alloy and a stack structure of APC alloy and ITO (ITO/APC/ITO). The APC alloy is an alloy of silver (Ag), palladium (Pd) and copper (Cu).

In the top-emission structure, the common electrode 173 may be formed of a transparent conductive material (TCP) such as ITO and IZO that can transmit light, or a semi-transmissive conductive material such as magnesium (Mg), silver (Ag) and an alloy of magnesium (Mg) and silver (Ag). When the common electrode 173 is formed of a semi-transmissive metal material, the light extraction efficiency can be increased by using microcavities.

The encapsulation layer TFTL may be disposed on the common electrode 173. The encapsulation layer TFTL includes at least one inorganic layer to prevent permeation of oxygen or moisture into the emission material layer EML. In addition, the encapsulation layer TFTL includes at least one organic layer to protect the emission material layer EML from foreign substances such as dust. For example, the encapsulation layer TFTL may include a first inorganic encapsulation layer TFE1 and an organic encapsulation layer TFE2. The first inorganic encapsulation layer TFE1 may be disposed on the common electrode 173, the organic encapsulation layer TFE2 may be disposed on the first inorganic encapsulation layer TFE1, and the second inorganic encapsulation layer TFE3 may be disposed on the organic encapsulation layer TFE2.

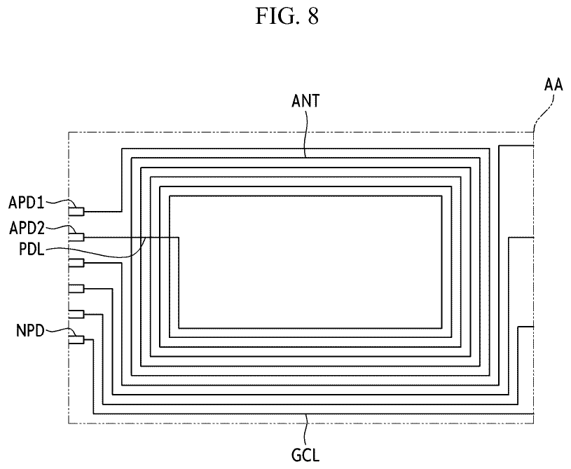

FIG. 8 is a block diagram showing an antenna arrangement structure of area AA shown in FIG. 3.

Referring to FIG. 8, the antenna ANT is patterned in the antenna placement area 3HD of the display panel 100 in a zigzag shape, a circular shape, a coil shape, a polygonal block shape, or a combination of preset blocks and lines on a plane, thereby transmitting electromagnetic waves containing pulse wave signals and electric signals related to biometric information. In addition, the antenna ANT may be patterned in an irregularly shaped pattern, such as a zigzag pattern or a maze shape, in the antenna placement area 3HD to transmit electromagnetic waves.

One end of the antenna ANT may be electrically connected to a first signal pad APD1, and the other end of the antenna ANT may be connected to a second signal pad APD2 through at least one contact hole and a connection line PDL.

In addition, in the antenna placement area 3HD, at least one data and gate signal line GCL and at least one third signal pad NPD may be further formed.

The antenna ANT formed in the antenna placement area 3HD may be formed through at least one patterning process in one of the same process layers during the manufacturing process, and the connection line PDL may be formed on a different process layer from the antenna ANT and electrically connected to the antenna ANT through at least one contact hole.

The wireless communication module 400 may sequentially transmit the electric signals in respect to the pulse wave signals and the biometric information to at least one signal pad APD1 and APD2 electrically connected to the antenna ANT, so that the antenna ANT may convert the electric signals in respect to the pulse wave signals and the biometric information to electromagnetic waves and transmit them wirelessly.

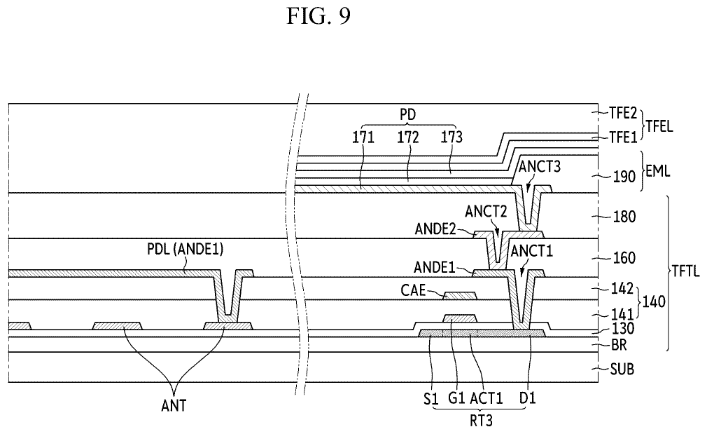

FIG. 9 is a cross-sectional view of a first embodiment showing a cross-sectional structure of a first light-sensing pixel and an antenna shown in FIG. 3.

Referring to FIG. 9, the antenna ANT may be formed through the same patterning process with the same metal material as a gate electrode G1 in the same manufacturing process layer as the gate electrode G1 of at least one transistor formed in the biological signal detection area 1HD.

Specifically, the antenna ANT may be patterned and formed with the same metal material through the same patterning processes as the gate electrode G1 of the transistor formed in the biological signal detection area 1HD on a gate insulating layer 130 in the antenna placement area 3HD. Accordingly, as same as the gate electrode G1 of the transistor, the antenna ANT may be made up of a single layer or multiple layers of one of molybdenum (Mo), aluminum (Al), chromium (Cr), gold (Au), titanium (Ti), nickel (Ni), neodymium (Nd) and copper (Cu) or an alloy thereof.

The connection line PDL of the antenna ANT may be patterned and formed with the same metal material through the same patterning process as a first anode connection electrode ANDE1 of the transistor formed in the biological signal detection area 1HD. Accordingly, the connection line PDL of the antenna ANT may be electrically connected to the antenna ANT through a first connection contact hole ANCT1 penetrating the gate insulator 130, the first interlayer-dielectric layer 141, and the second interlayer-dielectric layer 142.

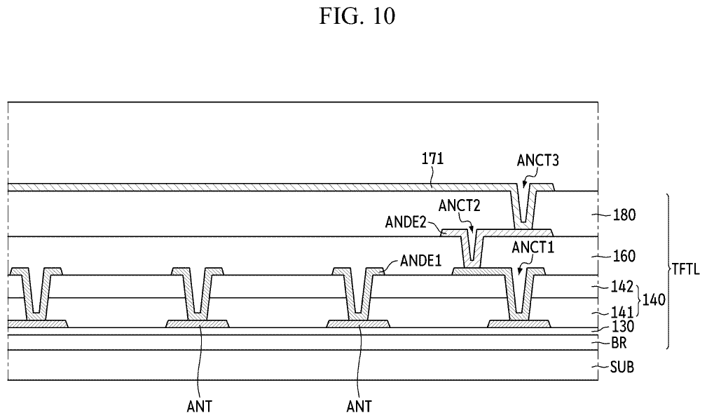

FIG. 10 is a cross-sectional view of a second embodiment showing a cross-sectional structure of a first light-sensing pixel and an antenna shown in FIG. 3.

Referring to FIG. 10, the antenna ANT may be formed with the same metal materials as the gate electrode and the first anode connection electrode ANDE1 of at least one transistor formed in the biological signal detection area 1HD through the same patterning process.

Specifically, the antenna ANT may be formed with the same metal material through the same patterning processes as the gate electrode and the first anode connection electrode ANDE1 of the transistor. Accordingly, the antenna ANT may be formed in the same manufacturing process layer as the gate electrode and the first anode connection electrode ANDE1 of the transistor. Accordingly, as the same as the gate electrode and the first anode connection electrode ANDE1 of the transistor, the antenna ANT may be made up of a single layer or multiple layers of one of molybdenum (Mo), aluminum (Al), chromium (Cr), gold (Au), titanium (Ti), nickel (Ni), neodymium (Nd) and copper (Cu) or an alloy thereof.

The connection line PDL of the antenna ANT may be formed with the same metal material through the same patterning process as a pixel electrode 171 of the transistor formed in the biological signal detection area 1HD. Accordingly, the connection line PDL of the antenna ANT may be electrically connected to the antenna ANT through the first and second anode connection electrodes ANDE1 and ANDE2 respectively formed in the first and second connection contact holes ANCT1 and ANCT2.

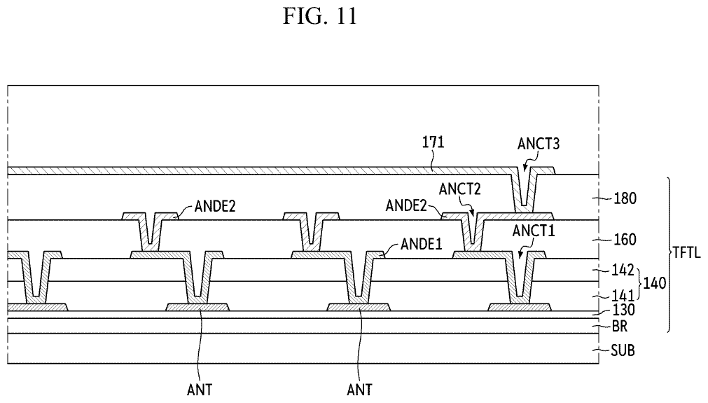

FIG. 11 is a cross-sectional view of a third embodiment showing a cross-sectional structure of a first light-sensing pixel and an antenna shown in FIG. 3.

Referring to FIG. 11, the antenna ANT may be formed with the same metal materials as the gate electrode G1, the first anode connection electrode ANDE1, and the second anode connection electrode ANDE2 of at least one transistor formed in the biological signal detection area 1HD through the same patterning process.

Specifically, the antenna ANT may be formed with the same metal material through the same patterning processes as the gate electrode, the first anode connection electrode ANDE1, and the second anode connection electrode ANDE2 of the transistor formed in the biological signal detection area 1HD. Accordingly, the antenna ANT may be formed in the same manufacturing process layer as the gate electrode G1, the first anode connection electrode ANDE1, and the second anode connection electrode ANDE2 of the transistor. Accordingly, as the same as the gate electrode G1 and the first and second anode connection electrodes ANDE1 and ANDE2 of the transistor, the antenna ANT may be made up of a single layer or multiple layers of one of molybdenum (Mo), aluminum (Al), chromium (Cr), gold (Au), titanium (Ti), nickel (Ni), neodymium (Nd) and copper (Cu) or an alloy thereof.

The connection line PDL of the antenna ANT may be formed of the same metal material as the pixel electrode 171 of the transistor formed in the biological signal detection area 1HD through the same patterning process. Accordingly, the connection line PDL of the antenna ANT may be electrically connected to the antenna ANT through the first and second anode connection electrodes ANDE1 and ANDE2 respectively formed in the first and second connection contact holes ANCT1 and ANCT2.

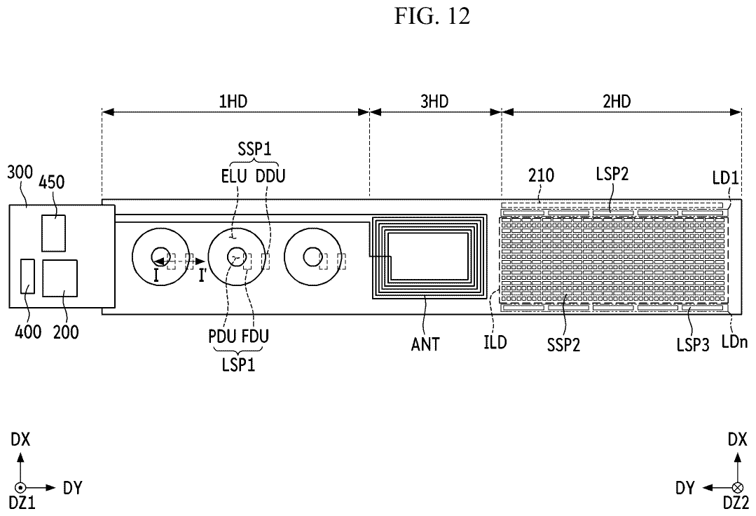

FIG. 12 is a top view showing the display panel shown in FIG. 2 that is spread out according to a second embodiment.

Referring to FIG. 12, an antenna placement area 3HD of the display panel 100 is disposed in an area between the biological signal detection area 1HD and the touch detection area 2HD.

The antenna ANT is patterned in a preset pattern in the antenna placement area 3HD. At this time, one end of the antenna ANT may be electrically connected to a first signal pad APD1, and the other end of the antenna ANT may be electrically connected to a second signal pad APD2 through at least one contact hole and a connection line PDL.

The antenna ANT may be patterned in a preset pattern such as a circular or polygonal coil pattern on a plane in the antenna placement area 3HD of the display panel 100 and transmit electromagnetic wave including electrical signals in respect to the pulse wave signals and the biometric information.

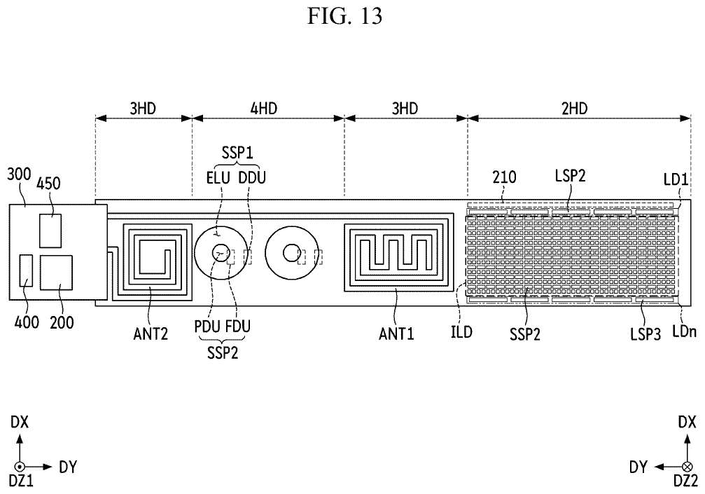

FIG. 13 is a top view showing the display panel shown in FIG. 2 that is spread out according to a third embodiment.

Referring to FIG. 13, an antenna placement area 3HD of the display panel 100 may be disposed so that a plurality of areas extend in an area between the connection area of the circuit board 300 and the biological signal detection area 1HD and in an area between the biological signal detection area 1HD and the touch detection area 2HD.

The antenna ANT may be patterned in a preset pattern in the antenna placement areas 3HD extending into a plurality of areas, and one end of the antenna ANT may be electrically connected to the first signal pad APD1. In addition, the other end of the antenna ANT may be electrically connected to the second signal pad APD2 through at least one contact hole and a connection line PDL.

Meanwhile, the antenna ANT may be divided into first and second antennas ANTI and ANT2 that transmit or receive electromagnetic waves of different frequencies. At this time, only the first antenna ANT1 may be patterned and formed in a preset pattern in one of the antenna arrangement areas 3HD extending into a plurality of areas. One end of such first antenna ANT1 may be electrically connected to the first signal pad APD1, and the other end of the first antenna ANTI may be electrically connected to the second signal pad APD2 through at least one contact hole and a connection line PDL.

In addition, a second antenna ANT2 that transmits or receives electromagnetic waves of a different frequency from the first antenna ANT1 may be patterned and formed in a preset pattern in at least one other antenna placement area 3HD among the antenna placement areas 3HD extending into a plurality of areas. One end of such second antenna ANT2 may be electrically connected to one other signal pad, and the other end of the second antenna ANT2 may be electrically connected to another one of the signal pads through at least one contact hole and a connection line. Here, the second antenna ANT2 may be patterned in a coil pattern, a zigzag pattern, or an irregular shape such as a maze shape, and be used as a coil or antenna for wireless charging of the battery 600.

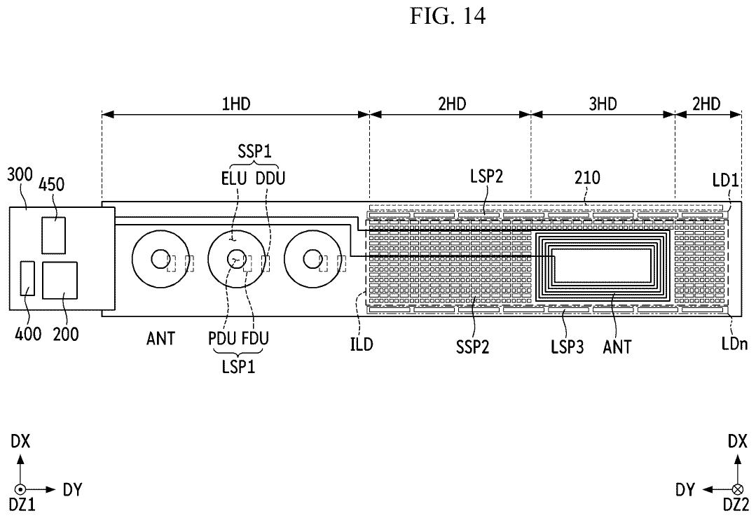

FIG. 14 is a top view showing the display panel shown in FIG. 2 that is spread out according to a fourth embodiment.

Referring to FIG. 14, the antenna placement area 3HD of the display panel 100 may be disposed in an area between the touch detection areas 2HD divided into a plurality of areas. Accordingly, the antenna ANT may be patterned with a preset pattern in the area between the touch detection areas 2HD divided into a plurality of areas, and one end of the antenna ANT may be electrically connected to the first signal pad APD1. In addition, the other end of the antenna ANT may be electrically connected to the second signal pad APD2 through at least one contact hole and a connection line PDL.

Meanwhile, the antenna placement area 3HD may be disposed in an area between the biological signal detection areas 1HD divided into a plurality of areas.

The antenna ANT may be patterned in a preset pattern in the area between the biological signal detection areas 1HD divided into a plurality of areas, one end of the antenna ANT may be electrically connected to the first signal pad APD1, and the other end of the antenna ANT may be electrically connected to the second signal pad APD2 through at least one contact hole and a connection line PDL.

The antenna ANT may be patterned in a preset pattern such as a circular or polygonal coil pattern on a plane in the antenna placement area 3HD of a preset display panel 100 and transmit electromagnetic wave including electrical signals in respect to the pulse wave signals and the biometric information.

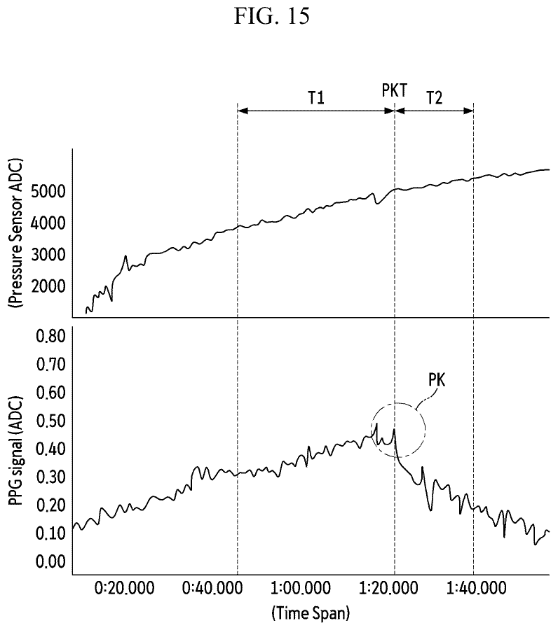

FIG. 15 is a graph for illustrating a method for calculating information about blood pressure among biometric information according to the embodiment.

Referring to FIG. 15, the blood ejected from the left ventricle of a heart a systole of a heart moves to the peripheral tissues, and accordingly the blood volume in the artery increases. In addition, red blood cells carry more oxygen in hemoglobin to the peripheral tissues during the systole of the heart. During a diastole of the heart, a part of the blood is sucked from the peripheral tissues towards the heart. When a peripheral blood vessel is irradiated with light, the irradiated light is absorbed by the peripheral tissue. The light absorbance is dependent on the hematocrit ratio and the blood volume. The light absorbance may have the maximum value in the systole of the heart and the minimum value in the diastole of the heart. Accordingly, the amount of light sensed by the light-sensing element PD may be the least during the systole of the heart and the most during the diastole of the heart.

In addition, when the user wears the wearable display device 10 in a blood pressure measurement mode, i.e., when the user brings her/his finger F into contact with the biological signal detection area 1HD, the pressure (contact pressure) applied to the pressure sensor of the sensing circuit 450 may gradually increase to reach the maximum value and then may gradually decrease. As the contact pressure increases, the blood vessels may constrict, resulting in small or zero blood flow rate. When the contact pressure decreases, the blood vessels dilate and blood begins to flow again. When the contact pressure further decreases, the blood flow rate increases more. Therefore, a change in the amount of light sensed by the first light-sensing pixel LSP1 may be proportional to a change in blood flow. Accordingly, the main driver circuit 200 generates pulse wave signals PPG according to the pressure applied by the user based on the pressure data value that is calculated by the pressure sensor and digitally converted (ADC of the pressure sensing unit) and the optical signal (PPG signal ratio) according to the amount of light sensed by the light-sensing element PD. The pulse wave signal PPG may have a waveform that oscillates according to heartbeat cycle.

The main driver circuit 200 may estimate blood pressures of the blood vessels of the finger F based on time differences between time points PKT corresponding to the peaks PK of the calculated pulse wave signals PPG and time points corresponding to the peaks of the filtered pulse waves. Specifically, the main driver circuit 200 may calculate pulse wave signals for predetermined time periods T1 and T2 before and after the time points PKT corresponding to the peaks PK of the calculated pulse wave signals and may detect the blood pressure according to differences between the pulse wave signals. Among the estimated blood pressures, the highest blood pressure may be calculated as the systolic blood pressure while the lowest blood pressure may be calculated as the diastolic blood pressure. In addition, other blood pressures such as the average blood pressure may be calculated using the estimated blood pressures.

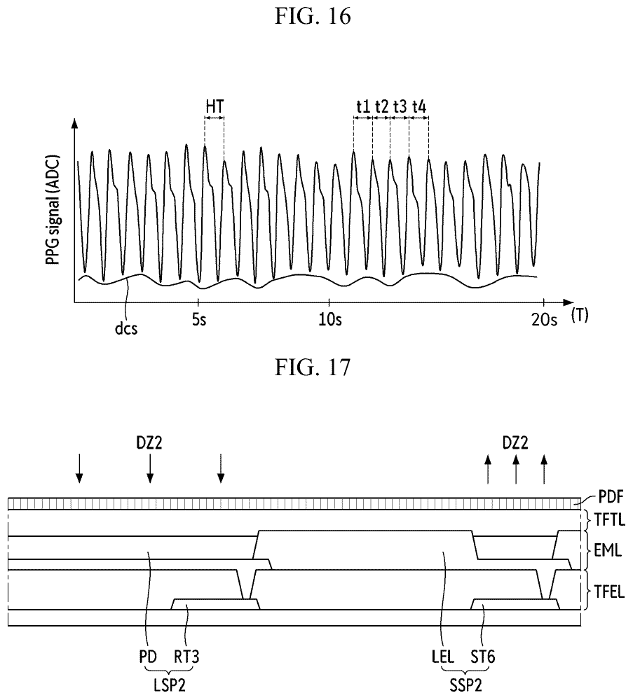

FIG. 16 is a graph for illustrating a method of calculating information about heart rate and respiration among biometric information according to an embodiment.

Referring to FIG. 16, the main driver circuit 200 sets the initial value or reference values for the pulse width f1 (e.g., systolic and diastolic period), the amplitude f2 (e.g., systolic blood pressure), the systolic pulse width f3 (e.g., systolic period) and the diastolic period f4 (e.g., diastolic pulse width) for each of the pulse wave signals (PPG). Subsequently, the main driver circuit 200 detects changes in the characteristics of pulse wave signals PPG input in real time compared to the initial values or reference values for the characteristics, i.e., changes in the systolic and diastolic period, the systolic blood pressure, the systolic pulse and the diastolic period, and builds a database with them as learning data. The main driver circuit 200 may output information on the blood pressure BP according to change rate or change size of the characteristics of the pulse wave signals PPG compared to the initial values or reference values of the characteristics.

The above-described method for measuring blood pressure is merely illustrative. A variety of methods may be employed such as ones disclosed in Korean Patent Laid-Open Publication Nos. 10-2018-0076050, 10-2017-0049280 and 10-2019-0040527, the entire contents of which are incorporated herein by reference.

The main driver circuit 200 may sample pulse wave signals during a predetermined sampling period before and after the time points PKT corresponding to the peaks PK of the pulse wave signals, and may detect generation period HT of high pulses for the sampled pulse wave signals PPG. In addition, the main driver circuit 200 may count the number of high pulses for each predetermined reference period (e.g., 60 seconds) for the sampled pulse wave signals PPG to detect biometric information about the heart period and heart rate HR.

In addition, the main driver circuit 200 detects the heart period HT and heart period changes t1 to t4 of the high pulses for each predetermined reference period for the peaks PK of the pulse wave signals, to detect heart rate variability (HRV) according to the rate of change of the heart period.

On the other hand, the main driver circuit 200 sequentially detects the generation period of low pulses and the magnitudes of the low pulses for the sampled pulse wave signals PPG. In addition, the main driver circuit 200 may detect the change period of the magnitudes des of the low pulses every predetermined reference period (e.g., 60 seconds) to detect changes in the respiration and respiratory rate RR of the user. In doing so, the main driver circuit 200 may analyze a period in which the magnitude des of low pulses rises and a period in which the magnitude des of low pulses falls, to detect the changes in the respiration and the respiratory rate RR of the user based on the periods in which the magnitude des of low pulses rises and falls.

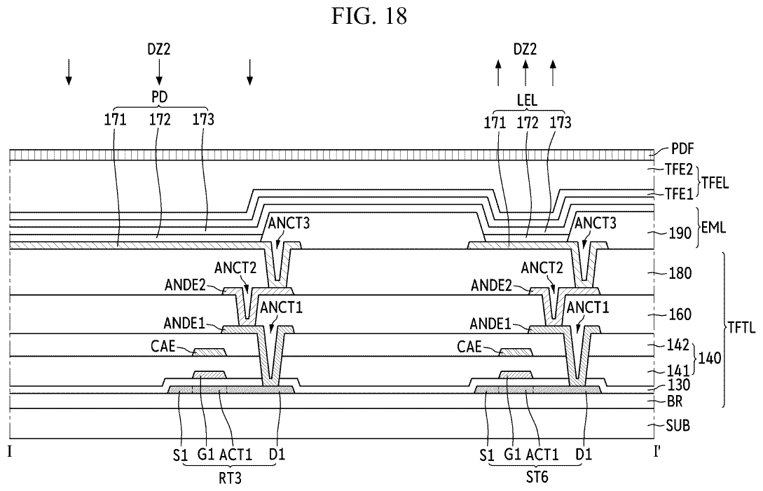

FIG. 17 is a cross-sectional view schematically showing the cross-sectional structure of a second light-emitting pixel and a second light-sensing pixel disposed in the touch detection area of FIG. 3. FIG. 18 is a cross-sectional view specifically showing the cross-sectional structure of the second light-emitting pixel and the second light-sensing pixel shown in FIG. 17.

Referring to FIGS. 17 and 18 in conjunction with FIG. 3, in the touch detection area 2HD of the display panel 100, a plurality of second light-emitting pixels SSP2 is disposed in vertical or horizontal stripes or in the PenTile matrix only in the light exit area ILD. Accordingly, the light-emitting elements of the second light-emitting pixels SSP2 arranged in a matrix in the light exit area ILD emit light toward the outside of the display panel 100 in a cylindrical shape, e.g., in the second direction DZ2 toward the outside of the cylindrical housing 500.

Meanwhile, a polarization plate layer PDF that causes light to enter or exit in a preset polarization direction may further be formed in the front direction of the touch detection area 2HD formed in the display panel 100. As the polarization plate layer PDF is formed in the front direction of the touch detection area 2HD, external light visibility characteristics and light emission efficiency can be improved.

On the other hand, the first light-sensing area LD1 is located on one side of the light exit area ILD, and only a plurality of second light-sensing pixels LSP2 is disposed in the first light-sensing area LD1. The light-sensing elements PD formed in the second light-sensing pixels LSP2 detects the amount of light reflected from a part of the user's body in the second direction DZ2 and generate a second light-sensing signal based on the detected amount of light.

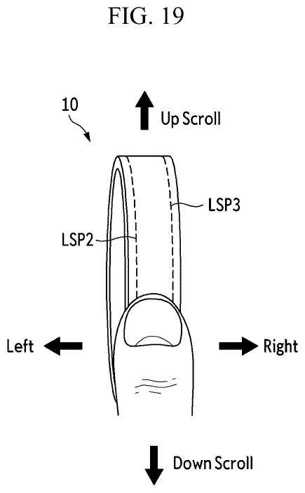

FIG. 19 is a view for illustrating a method for identifying the direction in which a user's touch moves on a wearable display device according to an embodiment.

Referring to FIG. 19, the second light-sensing pixels LSP2 formed in the first light-sensing area LD1 are arranged side-by-side on one side of the light exit area ILD along the longitudinal direction of the display panel 100 and the light exit area ILD.

The third light-sensing pixels LSP3 formed in the n^{th} light-sensing area LDn are arranged side-by-side on the opposite side of the light exit area ILD along the longitudinal direction of the display panel 100 and the light exit area ILD. Accordingly, the plurality of third light-sensing pixels LSP3 formed in the n^{th} light-sensing area LDn are arranged in parallel with the plurality of second light-sensing pixels LSP2 formed in the first light-sensing area LD1 with the light exit area ILD therebetween.

The main driver circuit 200 receives second light-sensing signals from the second light-sensing pixels LSP2 arranged side-by-side in the first light-sensing area LD1. In addition, the main driver circuit 200 receives third light-sensing signals from the third light-sensing pixels LSP3 formed in the n^{th} light-sensing area LDn.

The main driver circuit 200 determines the direction in which the user's touch moves based on changes in the magnitudes of the second and third light-sensing signals that are sequentially input from the second light-sensing pixels LSP2 formed in the first light-sensing area LD1 and the third light-sensing pixels LSP3 formed in the n^{th} light-sensing area LDn.

In concluding the detailed description, those skilled in the art will appreciate that many variations and modifications can be made to the preferred embodiments without substantially departing from the principles of the present disclosure. Therefore, the disclosed preferred embodiments of the disclosure are used in a generic and descriptive sense only and not for purposes of limitation.

What is claimed is:

1. A wearable display device comprising:
a housing formed in a ring shape or a cylindrical shape;
a display panel disposed in a ring shape or a cylindrical shape inside the housing, and configured to emit light in a first direction from an inner side of the display panel and receive light incident to the inner side to detect a first light-sensing signal and configured to emit light in a second direction from an outer side of the display panel and receive light incident to the outer side to detect a second light-sensing signal;
a main driver circuit configured to detect a pulse wave signal of a user and measure biometric information of the user by using the first light-sensing signal, and sense a movement of a touch by a part of the user's body by using the second light-sensing signal; and
a wireless communication module configured to transmit the biometric information of the user to a preset other mobile display device through antennas formed in a preset pattern in the display panel.

2. The wearable display device of claim 1,
wherein the display panel comprises:
a biological signal detection area in which, at least one first light-emitting pixel configured to emit the light in the first direction and at least one first light-sensing pixel configured to generate the first light-sensing signal depending on an amount of light reflected from a part of the user's body located in the first direction from the inner side of the display panel, are disposed;
a touch detection area in which, at least one second light-emitting pixel configured to emit the light in the second direction and at least one second light-sensing pixel configured to generate the second light-sensing signal depending on an amount of light reflected from a part of the user's body located in the second direction from the outer side of the display panel, are disposed; and
an antenna placement area in which the antennas are formed in at least one pattern shape among a zigzag shape, a circular shape, a coil shape, a polygonal block shape, or a combination of preset blocks and lines,
wherein the antenna placement area further comprises a plurality of signal pads connected to first ends and second ends of the antennas, respectively.

3. The wearable display device of claim 2,
wherein the wireless communication module sequentially is configured to transmit electric signals in respect to pulse wave signals and the biometric information of the user to at least one signal pad among the plurality of signal pads, and
the antennas are configured to convert the electric signals in respect to the pulse wave signals and the biometric information of the user into electromagnetic waves and transmit them.

4. The wearable display device of claim 2,
wherein the first end of an antenna among the antennas is electrically connected to a first signal pad among the plurality of signal pads, and the second end of the antenna among the antennas is electrically connected to a second signal pad among the plurality of signal pads through at least one contact hole and a connection line, and
the antennas are formed in a same layer, and the connection line is formed in a different layer from the antennas to be electrically connected to the antennas through at least one contact hole.

5. The wearable display device of claim 4,
wherein the antenna is formed in a same layer as a gate electrode of a transistor formed in the biological signal detection area with the same metal material as the gate electrode, and
the connection line includes a same metal material as a first anode connection electrode of a transistor formed in the biological signal detection area to be electrically connected to the antennas through a first connection contact hole penetrating a gate insulating film and first and second interlayer-dielectric films.

6. The wearable display device of claim 4,
wherein the antenna includes a same metal material as the gate electrode and the first anode connection electrode of the transistor formed in the biological signal detection area, and is formed in a same layer as the gate electrode and the first anode connection electrode, and
the connection line is formed of the same metal material as a pixel electrode of the transistor formed in the biological signal detection area, and is connected to the antennas through the first and second anode connection electrodes formed in the first and second connection contact holes, respectively.

7. The wearable display device of claim 4,
wherein the antenna includes a same metal materials as the gate electrode and the first and second anode connection electrodes of the transistor formed in the biological signal detection area, and is formed in a same layer as the gate electrode and the first and second anode connection electrodes, and
wherein the connection line includes a same metal materials as the pixel electrode of the transistor formed in the biological signal detection area, and is electrically connected to the antennas through the first and second anode connection electrodes formed in the first and second connection contact hole, respectively.

8. The wearable display device of claim 2,
wherein the antenna placement area is disposed in an area between the biological signal detection area and the touch detection area,
the first end of the antennas is electrically connected to a first signal pad among the plurality of signal pads, and the second end of the antennas is electrically connected to a second signal pad among the plurality of signal pads through at least one contact hole and a connection line, and
the antennas are formed in a same layer, and the connection line is formed in a different layer from the antennas to be electrically connected to the antennas through at least one contact hole.

9. The wearable display device of claim 2,
wherein the antenna placement area disposed so that a plurality of areas are extended in an area between a circuit board connection area of the display panel and the biological signal detection area and in an area between the biological signal detection area and the touch detection area,
the antennas are divided into first and second antennas configured to transmit or receive electromagnetic waves of different frequencies,
the first antenna is patterned and formed in at least one antenna placement area among the antenna placement areas extending to a plurality of areas, and
the second antenna is patterned and formed in at least the other antenna placement area among the antenna placement areas extending to a plurality of areas.

10. The wearable display device of claim 2,
wherein the antenna placement area is disposed in an area between touch detection areas divided into a plurality of areas, and
the antennas are patterned in a preset pattern in the area between the touch detection areas, the first end of the antenna is electrically connected to a first signal pad among the plurality of signal pads, and the second end of the antenna is electrically connected to a second signal pad among the plurality of signal pads through at least one contact hole and a connection line.

11. The wearable display device of claim 2,
wherein the at least one first light-sensing pixel disposed in the biological signal detection area comprises:
a light-sensing unit configured to output a light-sensing current corresponding to an amount of received light incident in the first direction; and
a sensing driver unit configured to supply the first light-sensing signal corresponding to an amount of light-sensing current to the main driver circuit,
wherein the light-sensing unit comprises a light-sensing element generating and outputting light-sensing current corresponding to the amount of received light,
and the light-sensing element senses light incident from the first direction, which is an inner direction of the display panel disposed in a cylindrical shape, to generate the light-sensing current.

12. The wearable display device of claim 11,
wherein the at least one first light-emitting pixel disposed in the biological signal detection area comprises:
a light-emitting unit configured to emit light of a green wavelength range; and
a pixel driver unit configured to apply a driving current to a light-emitting element of the light-emitting unit,
wherein the light-emitting unit is formed in a circular shape or a polygonal shape and emit light in the first direction.

13. The wearable display device of claim 12,
wherein the light-sensing element is formed in a circular shape or a polygonal shape to be formed in a shape surrounded by the light-emitting unit, and the light-emitting unit is formed in a circular shape or a polygonal shape to be formed in a shape surrounding the periphery of the light-sensing element.

14. The wearable display device of claim 2,
wherein the touch detection area comprises:
a light exit area comprising at least one second light-emitting pixel emitting light in the second direction, which is an outer direction of the housing and the opposite of the first direction;
a first light-sensing area disposed on one side of the light exit area, and comprising a plurality of second light-sensing pixels sensing an amount of light reflected from a part of the user's body in the second direction and generating second light-sensing signal depending on the amount of sensed light; and
an n^{th} light-sensing area disposed on the other side of the light exit area, and comprising a plurality of third light-sensing pixels sensing an amount of light reflected from a part of the user's body in the second direction and generating a third light-sensing signal depending on the amount of sensed light.

15. The wearable display device of claim 14,
wherein the plurality of second light-sensing pixels formed in the first light-sensing area are arranged side by side on one side of the light exit area along the longitudinal direction of the display panel and the light exit area,
the plurality of third light-sensing pixels formed in the n^{th} light-sensing area are arranged side by side on the other side of the light exit area along the longitudinal direction of the display panel and the light exit area, and
the plurality of third light-sensing pixels are arranged in parallel with the plurality of second light-sensing pixels with the light exit area disposed therebetween.

16. The wearable display device of claim 2,
wherein the touch detection area comprises a light exit area in which first unit pixels including a plurality of sub light-emitting pixels and at least one light-sensing pixel, and
the plurality of sub light-emitting pixels included in the first unit pixels emit light in the second direction, which is an outer direction of a display panel disposed in a cylindrical shape, and the at least one light sensing pixel senses light reflected from a part of the user's body in the second direction and generate the second light-sensing signal depending on the amount of sensed light.

17. The wearable display device of claim 16,
wherein the light exit area of the touch detection area further comprises a second unit pixel including a plurality of sub light-emitting pixels and one infrared light-emitting pixel.

18. A wearable electric display device comprising:
a housing formed in a ring shape or a cylindrical shape;
a display panel disposed in a ring shape or a cylindrical shape inside the housing, and configured to emit light in a first direction from an inner side of the display panel and receive light incident to the inner side to detect a first light-sensing signal and configured to emit light in a second direction from an outer side of the display panel and receive light incident to the outer side to detect a second light-sensing signal;
a main driver circuit configured to detect a pulse wave signal of a user and measure biometric information of the user by using the first light-sensing signal, and sense a movement of a touch by a part of the user's body by using the second light-sensing signal;
a wireless communication module configured to transmit the biometric information of the user to a preset other mobile display device through antennas formed in a preset pattern in the display panel;
a circuit board on which the main driver circuit is mounted, the circuit board electrically connected to the display panel;
a sensing circuit disposed in the housing or the circuit board to sense a direction of the user's movement and a pressure applied by the user's finger; and
a memory storing and transmitting the pulse wave signals of the user and the biometric information of the user.

19. An electronic device including a wearable display device,
wherein the wearable display device comprising:
a housing formed in a ring shape or a cylindrical shape;
a display panel disposed in a ring shape or a cylindrical shape inside the housing, and configured to emit light in a first direction from an inner side of the display panel and receive light incident to the inner side to detect a first light-sensing signal and configured to emit light in a second direction from an outer side of the display panel and receive light incident to the outer side to detect a second light-sensing signal;
a main driver circuit configured to detect a pulse wave signal of a user and measure biometric information of the user by using the first light-sensing signal, and sense a movement of a touch by a part of the user's body by using the second light-sensing signal; and
a wireless communication module configured to transmit the biometric information of the user to a preset other mobile display device through antennas formed in a preset pattern in the display panel.

20. An electronic device including a wearable display device,
wherein the wearable display device comprising:
a housing formed in a ring shape or a cylindrical shape;
a display panel disposed in a ring shape or a cylindrical shape inside the housing, and configured to emit light in a first direction from an inner side of the display panel and receive light incident to the inner side to detect a first light-sensing signal and configured to emit light in a second direction from an outer side of the display panel and receive light incident to the outer side to detect a second light-sensing signal;
a main driver circuit configured to detect a pulse wave signal of a user and measure biometric information of the user by using the first light-sensing signal, and sense a movement of a touch by a part of the user's body by using the second light-sensing signal;
a wireless communication module configured to transmit the biometric information of the user to a preset other mobile display device through antennas formed in a preset pattern in the display panel;
a circuit board on which the main driver circuit is mounted, the circuit board electrically connected to the display panel;
a sensing circuit disposed in the housing or the circuit board to sense a direction of the user's movement and a pressure applied by the user's finger; and
a memory storing and transmitting the pulse wave signals of the user and the biometric information of the user.
